(19) United States
(12) Patent Application Publication
(10) Pub. No.: US 2023/0279334 A1
(43) Pub. Date: Sep. 7, 2023

(54) A METHOD FOR THE PREPARATION OF FREEZE-DRIED QUALITY CONTROL PELLETS OF ESCHERICHIA COLI

(71) Applicant: Hainan Microkrypton Biotechnology Co., Ltd., Hainan (CN)

(72) Inventors: Wenxing LI, Hainan (CN); Yi WAN, Hainan (CN)

(21) Appl. No.: 17/598,408

(22) PCT Filed: Sep. 2, 2021
(86) PCT No.: PCT/CN2021/116086

§ 371 Date: Sep. 27, 2021

(30) Foreign Application Priority Data
Mar. 8, 2021 (CN) 202110250271.2

(51) Int. Cl. C12N 1/04; C12Q 1/10

(52) U.S. Cl. CPC C12N 1/04; C12Q 1/10; C12R 2001/19

(57) ABSTRACT

The present invention provides a method for the preparation of quality control pellets freeze-dried of Escherichia coli, including steps such as making puree containing Escherichia coli, making freeze-drying protective agent, mixing freeze-drying protective agent with bacterial puree and freeze-drying to make freeze-dried powder, and finally making quality control pellets with high activity.

### TECHNICAL FIELD

The present invention relates to an activity controllable preservation method for microorganisms, specifically a process in which Escherichia coli is freeze-dried to produce quality control pellets.

### BACKGROUND

Foodborne contamination includes 31 different types such as microorganisms, viruses, toxins, parasites as well as chemicals, etc. Foodborne pollution causes hundreds of clinical diseases, and the annual economic losses due to foodborne pollution worldwide are up to several hundred billion. Among these factors, microbial contamination is one of the main factors of foodborne pollution, which seriously affects national life health and economic safety, and carrying out research on detection and control of pathogenic microorganisms is of great significance for national economic construction and health management.

Common detection methods for pathogenic microorganisms include plate culture, agglutination assay, molecular amplification, and photo electric sensing based on the metabolic properties of microbial growth, which have been developed into classical methods and have achieved extremely wide applications. However, with the exception of plate culturing, which enables accurate quantification of pathogenic microorganisms in an absolute quantitative manner, other assays are semi quantitative. Therefore, QC prior to the detection of pathogenic microorganisms is an extremely important loop for all semi quantitative methods or equipment, and QC of testing equipment and methods is an important prerequisite to ensure the effectiveness of the equipment and methods. This has implications for the accuracy of the results obtained for the detection of pathogenic microorganisms and for the precision of the assays between different methods, different equipment, or even different machines of the same equipment. The quality control by microbiological detection methods or equipment can effectively improve the accuracy and precision, which can better and more accurately judge the situation of food hygiene and make a correct evaluation of the degree of contamination with pathogenic microorganisms of food, and can also provide an accurate basis for food hygiene related quality control work.

Common detection methods for pathogenic microorganisms include plate culture, agglutination assay, molecular amplification, and photo electric sensing based on the metabolic properties of microbial growth, which have been developed into classical methods and have achieved extremely wide applications. However, with the exception of plate culturing, which enables accurate quantification of pathogenic microorganisms in an absolute quantitative manner, other assays are semi quantitative. Therefore, QC prior to the detection of pathogenic microorganisms is an extremely important loop for all semi quantitative methods or equipment, and QC of testing equipment and methods is an important prerequisite to ensure the effectiveness of the equipment and methods. This has implications for the accuracy of the results obtained for the detection of pathogenic microorganisms and for the precision of the assays between different methods, different equipment, or even different machines of the same equipment. The quality control by microbiological detection methods or equipment can effectively improve the accuracy and precision, which can better and more accurately judge the situation of food hygiene and make a correct evaluation of the degree of contamination with pathogenic microorganisms of food, and can also provide an accurate basis for food hygiene related quality control work.

In terms of current detection of pathogenic microorganisms, the detection targets and quantitative parameters adopted by different methods are not the same. Such as molecular experiments, different detection agencies may choose different target fragments of microorganisms or use different primers for analysis, so the judgment of the different results obtained is also quite different; For example, microbial photodetectors, now the market of microbial photodetectors is a large number and variety of different kinds, such as tempo detection, bactrac detection, soleris detection system, pn-ins32 type detection system, BIOLOG microbial identification system and Bax system Q7 detection system, etc., the strategies of photoelectric sensing also have turbidity, ultraviolet absorbance, fluorescence, etc, What is more about the results between different instrument devices is the inability to place judgment under the same criteria. Freeze drying preservation of microorganisms is a common microbial preservation method, but in practical applications, the freeze-drying process often causes uncontrollable microbial damage, which leads to uncontrollable loss of activity. While suitable freeze-drying protectants can effectively alleviate this problem, therefore, screening for suitable freeze-drying protectants is an important loop in microbial preservation, and suitable freeze-drying protectants are directly related to the control of microbial activity loss rate and finally the control of the number of strains in the resulting quality control pellets.

In view of the above status, it is obvious to see the importance of quality control of pathogen microorganism detection, in order to make the test results comparable, uniform live Micro Organism control pellets and methods are also of great importance.

### SUMMARY OF THE INVENTION

To solve the above problems, the object of the present invention to provide a method for preparing freeze-dried quality control pellets of Escherichia coli, the use of a special freeze-dried protective agent makes the quality control pellets highly active and convenient to store and use. The later testing standards are unified and the testing results are accurate. To achieve the above purposes, the technical means taken by the invention are:

A method for the preparation of Escherichia coli freeze-dried quality control pellets comprising the following steps: S1 making bacterial mud containing E. coli: using nutrient broth culture to obtain the bacterial fluid that got E. coli, making the bacterial mud for later use after aliquoting.

Step S1 includes:
S11 aliquot the obtained broth as 1 ml / tube;
S12 aliquot the completed bacterial fluid was sequentially centrifuged to remove the supernatant;
S13 add 1.0×PBS as wash solution, repeat centrifugation and remove supernatant;
S14 remove the medium and metabolites in it, and centrifuge treatment again to get the bacterial mud.

Further, the broth was kept at 8000 rpm for 10 min when centrifugation was performed in S12, S13, and S14.

S2 to make the freeze-drying protectant: a predetermined amount of water-soluble sugars, proteins, surfactants, inorganic salts, and antioxidants, respectively, were added to the sterile water and mixed by shaking to obtain a homogeneous aqueous solution as the freeze-drying protectant for use.

Among them, the mass concentration fraction of each component in the lyoprotectant is: 2-5% trehalose, 5-8% nonfat dry milk, 2-3% glycerol, 4-5% inorganic salts, 0.05-0.1 % antioxidant, and the rest is sterile water. Preferably, the mass concentration fraction of each component in the freeze-drying protectant is: 2% trehalose, 8% nonfat dry milk, 2% glycerol, 5% inorganic salt, 0.05% antioxidant, and the rest is sterile water.Of these, the inorganic salts are any one of sodium glutamate, sodium chloride, sodium sulfate, potassium chloride, ammonium chloride, calcium carbonate. The antioxidants are any of vitamin C, vitamin E, sodium sulfite. Preferably, the inorganic salt is sodium glutarate; The antioxidant described is vitamin E.

S3 mixed: to the mash of step S1 added the freeze-drying protecting agent made in step S2, in which the volume ratio of freeze-drying protecting agent: mash was 1:10, mixed to obtain a mixture solution evenly;

S4 freeze and drying: after subjecting the mixture prepared from step S3 to freeze treatment, transfer to freeze dryer and dry thoroughly to obtain quality control pellets for storage.

Among them, freezing treatment refers to freezing the mixture liquid at an environment of - 80° C. for 5 h.

Quality control pellets prepared by the above mentioned preparation method are used for the quality control of different testing equipment or detection methods after resuspension by adding normal saline or other solutions that are non-destructive to the activity of Escherichia coli. The use of this quality control pellet provides assurance that the assay samples are uniform and that the assay results are accurate and comparable.

The beneficial effects of the present invention are:

Aiming at this pathogenic microbial model of E. coli, a freeze-dried protective agent capable of efficiently preserving the activity of E. coli was identified from such components as proteins, water-soluble sugars, surfactants, inorganic salts as well as antioxidants. Based on this freeze-drying protector, quality control pellets of E. coli with high activity are made using freeze-drying method for use in quality control based on different detection methods or detection equipment. In this invention, the water-soluble sugar is trehalose and the protein is nonfat dry milk, which is able to make the effect of freeze-drying protector optimal, and the surfactant is glycerol, which is able to elevate the activity of E. coli in quality control pellets.

The present invention screens components and contents of a suitable freeze-drying protector for the nondestructive preservation of the activity of E. coli and, in turn, the preparation of quality control pellets of E. coli. And the activity loss of quality control pellets prepared by the present invention during the preparation process can be calculated, convenient to guide the preparation of quality control pellets with specific strain number requirements in subsequent applications.

The present invention may be used for the preparation of freeze-dried quality control pellets for other pathogenic microorganisms in addition to quality control pellet preparation for E. coli, providing standards for accurate pathogenic microorganism detection.

### BRIEF DESCRIPTION OF FIGURES

FIG. 1a shows the real-time growth curves obtained under the forbid instrument for E. coli at different concentrations;

FIG. 1b the standard curve derived from FIG. 1A statistics;

FIG. 2a is the real-time growth curve obtained under the forbid instrument for E. coli quality control pellets after resuspension and dilution to different concentrations;

FIG. 2b the standard curve resulting from FIG. 2A statistics;

FIG. 3 is a consistency analysis between the theoretical number of strains in the quality control pellet and the number of strains measured from the standard curve;

FIG. 4 for the real-time growth curve contrast plots obtained using different sugars vs. using trehalose in example 2 of the present invention;

FIG. 5 real time growth curve contrast plots obtained for example 3 of the present invention using different contents of trehalose;

FIG. 6 plots of real-time growth curves versus that obtained for example 4 of the present invention using different contents of Non Fat dry milk;

FIG. 7 for the real-time growth curve contrast plots obtained in example 5 of the present invention with and without the addition of glycerol to the freeze-drying protectant;

FIG. 8 for the real-time growth curve contrast plots obtained for example 6 of the present invention using different contents of glycerol;

FIG. 9 for the real-time growth curve versus plot obtained for the salt like substances of different compositions in Example 7 of the invention;

FIG. 10 for the real-time growth curve versus plot obtained for example 8 of the present invention using different contents of sodium glutarate;

FIG. 11 for the real-time growth curve contrast plots obtained for example 9 of the present invention with or without different antioxidants.

### DETAILED DESCRIPTION

The present invention provides a method for preparing freeze-dried quality control pellets of Escherichia coli, which comprises the following steps:

S1 making bacterial mud containing E. coli: using nutrient broth to grow bacteria to obtain Escherichia coli bacteria liquid, the method of cultivation and multiplication persons skilled in the art choose the conventional way, which is not the focus of the present invention, and is not described herein. The obtained bacterial fluid was aliquoted as 1 ml / tube, and the aliquot completed bacterial fluid was sequentially centrifuged and cleaned.

For centrifugation, after holding the broth in place, start the centrifuge and gradually increase the speed, after 6 s when the speed reaches 8000 rpm, keep the broth at that speed for 10 min

Lysates after centrifugation were used 1×Washing treatments were performed with PBS, which is a commercially available product: sigma Aldrich, cat. No: p5493.

Remove supernatant after wash treatment, repeat centrifugation and wash treatment one more time and remove supernatant.

After that, the medium and metabolites in the bacterial fluid were removed and treated again by centrifugation to obtain the bacterial sludge containing E. coli.

S2 to make the freeze-drying protecting agent: a predetermined amount of water-soluble sugars, proteins, surfactants, inorganic salts, and antioxidants, respectively, Add the above components into sterile water and mix uniformly to obtain an aqueous solution as a freeze-drying protective agent for use; Among them, the mass concentration fraction of each component in the lyoprotectant is: 2-5% trehalose, 5-8% nonfat dry milk, 2-3% glycerol, 4-5% inorganic salts, 0.05-0.1 % antioxidant, and the rest is sterile water.

Of these, the inorganic salts are any one of sodium glutamate, sodium chloride, sodium sulfate, potassium chloride, ammonium chloride, calcium carbonate.The antioxidants are any of vitamin C, vitamin E, sodium sulfite.

Preferably, the mass concentration fraction of each component in the lyoprotectant is 2-5% trehalose, 5-8% nonfat dry milk, 2-3% glycerol, 4-5% sodium glutarate, 0.05-0.1 % vitamin E, and the rest is sterile water.

S3 mix: to the mash of step S1 add the freeze-drying protecting agent made in step S2 in which the volume ratio of freeze-drying protecting agent: mash is 1:10, in this invention, using 100 µL of freeze-drying protecting agent was added to each tube and mixed well to obtain a mixture.

S4 freeze and drying: the mixture prepared from step S3 was frozen at-80° C. for 5 h and after this freezing treatment transferred to a freeze dryer for at least 24 h to obtain a quality control pellet of E. coli after thorough drying and storage.

Quality control pellets require close storage at 4° C.

The quality control pellets prepared by the invention, when used, are used for the quality control of different testing equipment or detection methods by plotting a standard curve for accurate quantification of the E. coli content within them after resuspension by addition of normal saline or other solutions that are non-destructive to the E. coli activity.The use of the quality control pellets of the present invention can guarantee that the detection samples are uniform, ensuring that the detection results are accurate and comparable.

### Example 1

In this example, a method for the preparation of quality control pellets freeze-dried for Escherichia coli, comprising the following steps:

S1 making bacterial sludge containing E. coli: using nutrient broth culture to obtain bacterial fluid containing E. coli, the obtained bacterial fluid was divided into 1 ml / tube, the aliquot of the completed bacterial fluid was carried out in accordance with the steps described previously. After several centrifugation and washing treatments sequentially, the bacterial sludge containing E. coli was obtained.

S2 to make the freeze-drying protecting agent: a predetermined amount of water-soluble sugars, proteins, surfactants, inorganic salts, and antioxidants, respectively, was added to the sterile water and mixed to obtain an aqueous solution evenly as the freeze-drying protecting agent for use;

The mass concentration fractions of each component in the lyoprotectant were as follows: 2% trehalose, 8% nonfat dry milk, 2% glycerol, 5% sodium glutarate, 0.05% vitamin E, and the rest sterile water.

S3 mixed: to the mash of step S1 add the freeze-drying protecting agent made at step S2, in this example, add 100 to each tubeµL of freeze-drying protector, mix well to obtain a blend;

S4 freeze and drying: freeze the mixture prepared from step S3 at - 80° C. for 5 h and after this freezing process transfer to a freeze dryer to obtain quality control pellets after thorough drying.

When in use, the prepared quality control pellets were resuspended in 1 ml of normal saline, diluted with normal saline to different concentration gradients, and subsequently transferred to 5 ml of Escherichia coli medium containing 0.075 g of 4-methylumbelliferone-β-D-glucuronide, a glucuronidase secreted by Escherichia coli acts on 4-methylumbelliferone-β-D glucuronides, causing them to fluoresce for real-time monitoring of microbial growth curves. The time at which the fluorescence reached the threshold was recorded to determine the log linear correlation of the E. coli concentration with the time to reach the threshold, which was used as a standard curve for further quantitative analysis of the E. coli numbers in real samples.

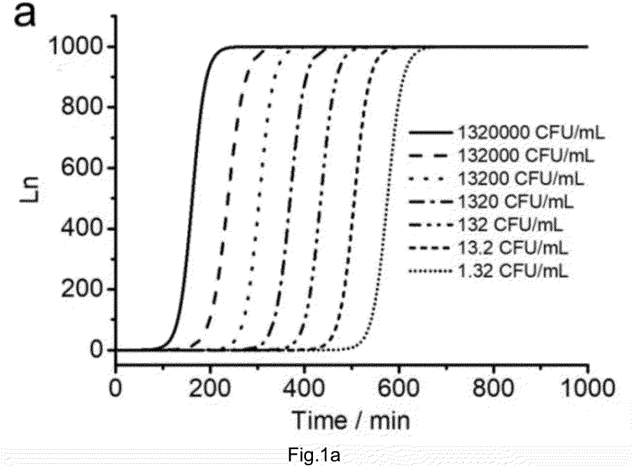

As shown in FIG. 1a and FIG. 1b, FIG. 1a is the real-time growth curve obtained for E. coli on fluorescence photo biosensor (FORBID) at different concentrations, where abscissa is the growth time, ordinate is the fluorescence signal intensity, and the number of E. coli is the number of viable strains obtained by plate counting;FIG. 1b is the standard curve obtained after the statistics of FIG. 1a, where the abscissa is the number of strains and the ordinate is the corresponding time when the fluorescence intensity of FIG. 1A reached a certain value, this curve was used for subsequent counting of active E. coli in quality control pellets. Real time amplification curves of liquor samples with different strain concentrations on a fluorescent photo biosensor (forbid) and the log linear correlation relationship of strain concentration and the time when the fluorescent signal reached the monitoring point.

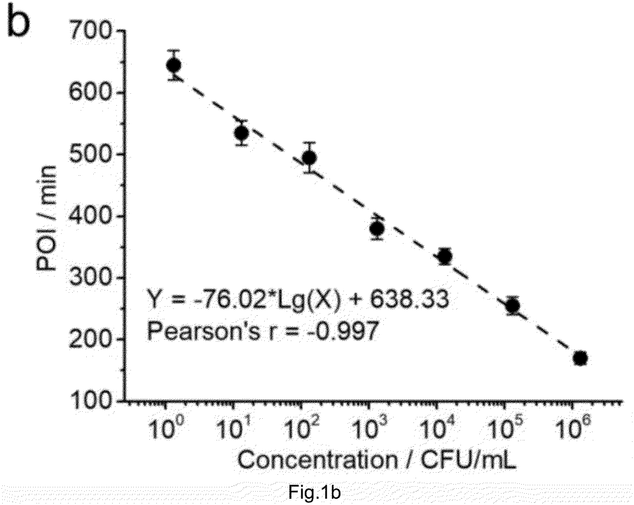

FIG. 2a is the real-time growth curve obtained under the FORBID instrument after the E. coli quality control pellets were resuspended and diluted to different concentrations, the abscissa is the growth time, the ordinate is the fluorescence signal intensity, and the E. coli number is the theoretical number of the obtained living strains calculated; FIG. 2b is the standard curve obtained after the statistics of FIG. 2a, where the abscissa is the number of strains and the ordinate is the corresponding time when the fluorescence intensity of FIG. 2a reaches a certain value. The real-time amplification curves on the fluorescence photo biosensor (FORBID) after the quality control pellets were resuspended and diluted to different concentrations and the log linear correlation of the strain concentration and the time that the fluorescence signal reached the monitoring point could be obtained from the above two plots. The growth and standard curves obtained with the quality control beads were highly consistent with those of the normal strain, indicating that the prepared quality control beads are capable of being used in the forbid instrument and other methods that target Escherichia coli for detection and the instrument’s quality control.

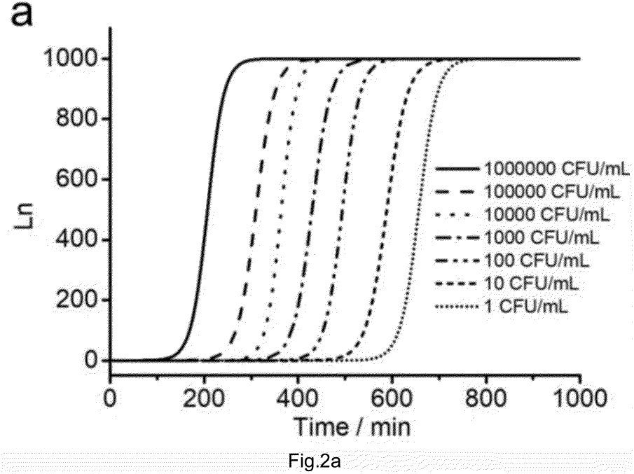

FIG. 3 is a consistency analysis of the theoretical number of strains in the quality control pellet with the number of strains measured from the standard curve. The an route abscissa is the theoretical live E. coli number after resuspension and dilution of the quality control pellet, and the ordinate is the actual live E. coli number after calculation by the FORBID instrument with the standard curve of FIG. 1b.The number theoretically calculated is highly consistent with the number actually detected, indicating that the quality control pellets prepared by the present invention can be used for quality control of the detection method versus the detection instrument. In addition, the consistency of the data measured from six different quality control pellets is good, which indicates that the rate of loss of Escherichia coli activity in the quality control pellets prepared in this method is essentially controllable, further indicating that the quality control pellets prepared in this method have value for use in quality control.

The present invention focuses on determining the most appropriate freeze-drying protector components and contents for use in the preparation of quality control pellets to ensure high activity of E. coli and computability of loss of activity during preparation. Therefore, the subsequent examples and ratios are listed only with the main differences, other conditions and manufacturing process are the same, and are not described.

### Example 2

Multiple sets of data contrasts were made in the present examples, differing from example 1 by: 6% trehalose, 6% sucrose, 6% lactose, 6% maltose, or 6% glucose, respectively, in 10% nonfat dry milk. The control group was centrifuged and rinsed but the strain was not frozen.

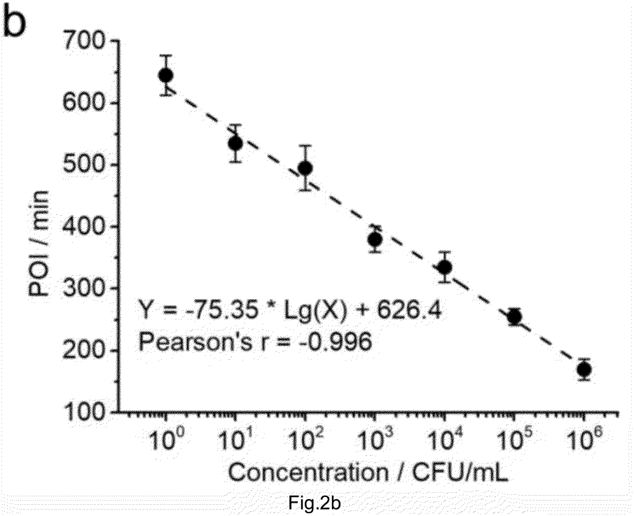

The results obtained from testing the quality control pellets prepared above are shown in FIG. 4. lt is known from the Fig. that at 6% trehalose, more E. coli living strains were obtained than with other sugars.

### Example 3

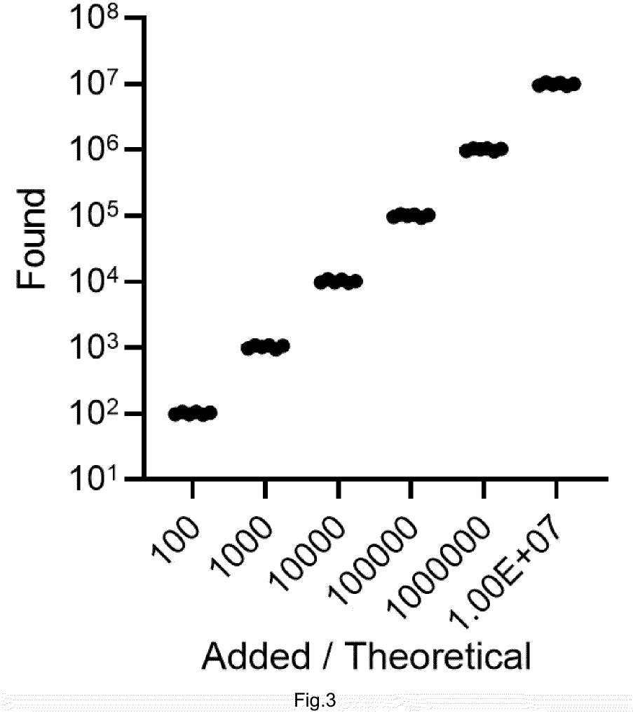

The present example differs from example 1 in that each component in the freeze-drying protectant contains 1%, 2%, 4%, 6%, 8%, or 10% trehalose in 10% nonfat dry milk, respectively, as the mass concentration fraction of each component. The results obtained from testing the quality control pellets prepared above are shown in FIG. 5, and the results showed that 10% nonfat dry milk + 2% trehalose had the best preservation effect on the activity of the strains.

### Example 4

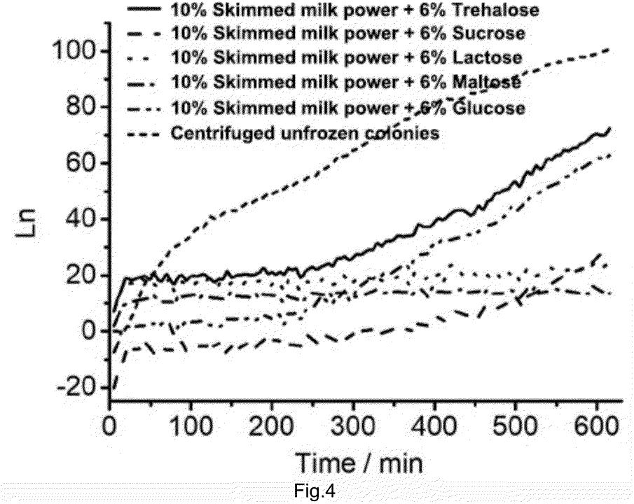

This example differs from example 1 in that each of the mass concentration fractions of each component in the freeze-drying protectant contains 8%, 10%, 12%, 14%, 16%, 18%, or 20% nonfat dry milk in 2% trehalose. The examination results of the quality control pellets prepared above were shown in FIG. 6, and the results showed that 8% nonfat dry milk + 2% trehalose had the best preservation effect on the activity of the strains.

### Example 5

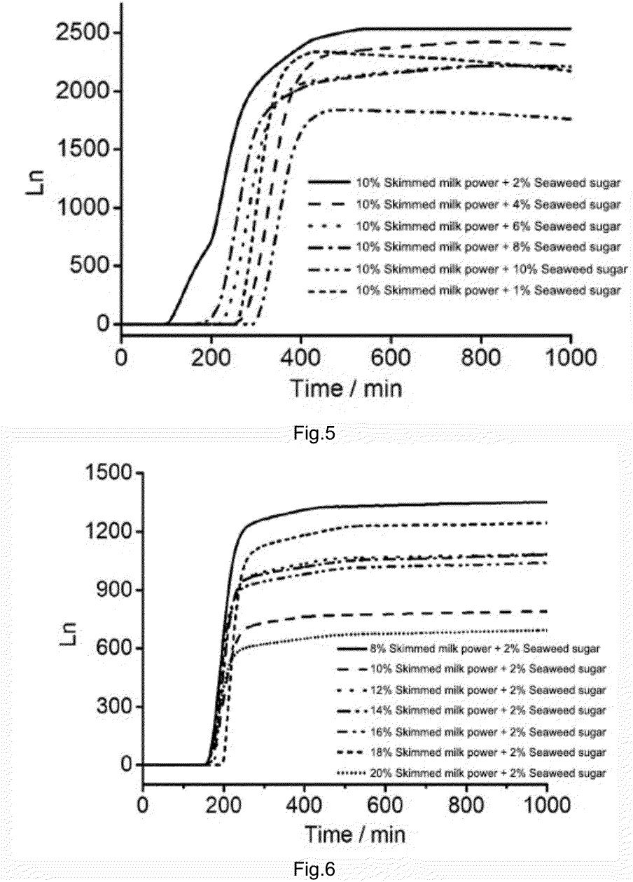

The difference in the present example from example 1 is that the mass concentration fraction of each component in the freeze-drying protectant contained 1% glycerol or not in 10% nonfat dry milk, 1% trehalose, respectively. The results of the tests performed on the quality control pellets prepared above are shown in FIG. 7, and the results showed that the strain activity was better preserved with 1% glycerol.

### Example 6

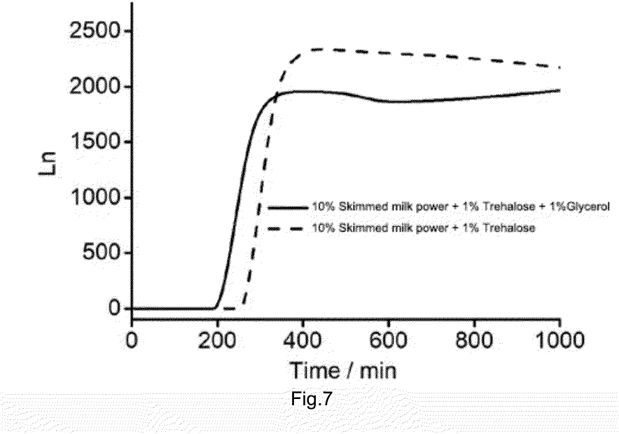

The difference from example 1 in this example is that the mass concentration fraction of each component in the lyoprotectant contained 0%, 1%, 2%, 3%, 4%, or 5% glycerol in 10% nonfat dry milk, 2% trehalose, respectively. The examination results of the quality control pellets prepared above are presented in FIG. 8, and the results showed that 10% nonfat dry milk + 2% trehalose + 2% glycerol had the best effect on the preservation of strain activity.

### Example 7

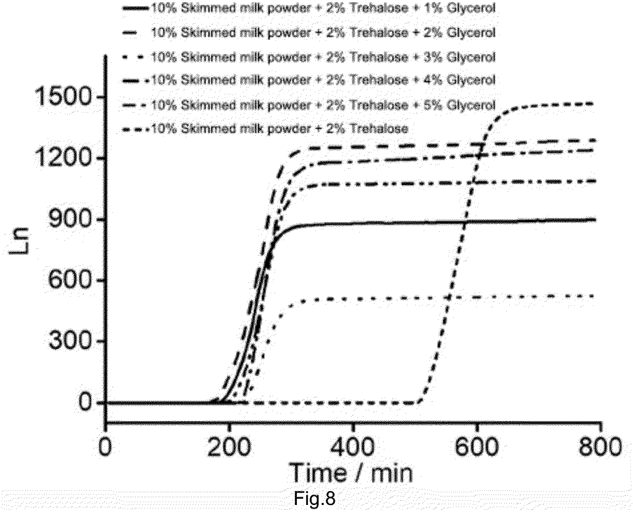

The difference in this example from example 1 is that the mass concentration fraction of each component in the lyoprotectant contained no salt in 10% nonfat dry milk, 1% trehalose with or without 1% sodium chloride, 1% glutamine, 1% sodium sulfate, 1% potassium chloride, 1% ammonium chloride, 1% calcium carbonate, or none. The examination results of the quality control pellets prepared above were shown in FIG. 9, and the results showed that 10% nonfat dry milk + 1% trehalose + 1% sodium glutarate had the best preservation effect on the activity of the strains.

### Example 8

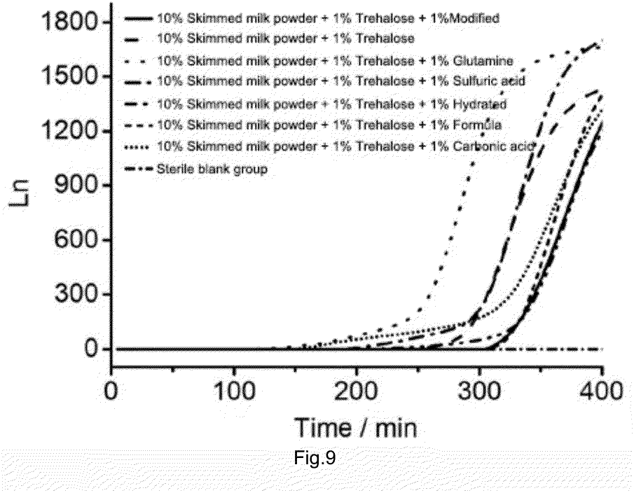

The difference from example 1 in this example is that the mass concentration fraction of each component in the lyoprotectant contained 1%, 2%, 3%, 4%, 5%, 6%, or no sodium glutarate in 10% nonfat dry milk, 2% trehalose, respectively. The test results of the quality control pellets prepared above were shown in FIG. 10, and the results showed that 10% nonfat dry milk + 2% trehalose + 5% sodium glutarate had the best preservation effect on the activity of the strains.

### Example 9

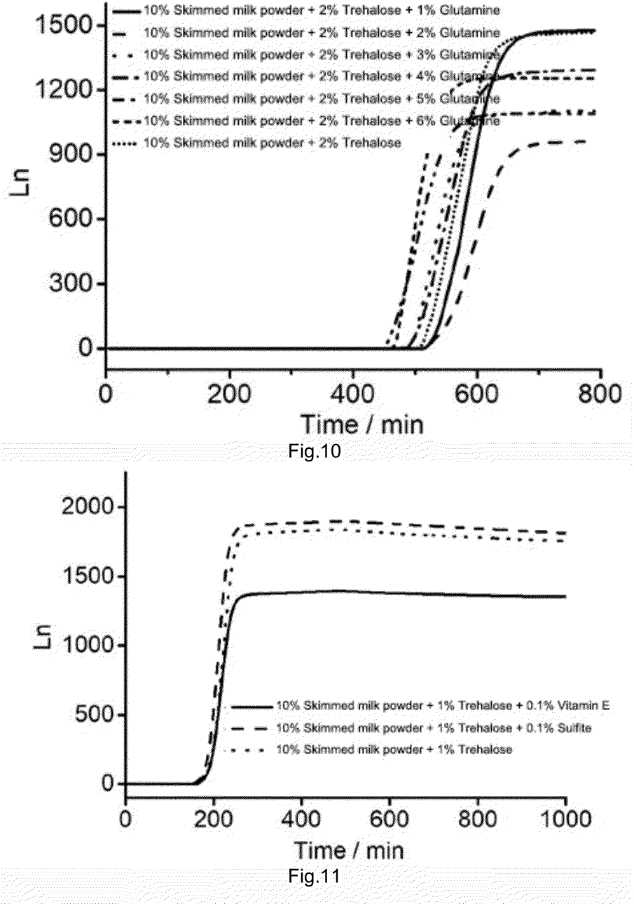

The difference in the present example from example 1 is that the mass concentration fraction of each component in the freeze-dried protectant contained 0.1 % vitamin E, 0.1% sodium sulfite or no antioxidant in 10% Non Fat dry milk, 1% trehalose respectively. The results of the tests performed on the above-mentioned quality control pellets are presented in FIG. 11, and the results showed that the effect of antioxidants on the preservation of strain activity was not obvious. However, because antioxidants are favorable for the long-term preservation of strains, vitamin E was selected to be added as an antioxidant to the protective agent, in addition, 1% vitamin E will affect the strain morphology, and 0.05% will not, so 0.05% vitamin E was finally selected to be added to the protective agent.

In the present invention, suitable components of the freeze-drying protector were screened for their content with non-destructive preservation for the activity of E. coli.

1. Preparation method of freeze-dried quality control pellets of Escherichia coli, which is characterized in that it comprises the following steps:
S1 to make bacterial mud containing E. coli: using nutrient broth culture to obtain bacterial fluid to obtain E. coli, after aliquot to make bacterial mud for use;
S2 to make the freeze-drying protecting agent: a predetermined amount of water-soluble sugars, proteins, surfactants, inorganic salts, and antioxidants were separately taken, and the above components were respectively added to sterile water and mixed by shaking to obtain an aqueous solution evenly to be used as the freeze-drying protecting agent;
S3 mixed: Add the freeze-dried protective agent made in step S2 to the bacterial mud in step S1, in which the volume ratio of freeze-drying protecting agent: mash was 1:10, mixed to obtain a mixture solution evenly;
S4 freeze and drying: after subjecting the mixture prepared from step S3 to freeze treatment, transfer to freeze dryer and dry thoroughly to obtain quality control pellets for storage.

2. Preparation method of freeze-dried quality control pellets of Escherichia colias recited in claim 1, step S1 includes:
S11 aliquot the obtained broth as 1 ml / tube;
S12 aliquot the completed bacterial fluid was sequentially centrifuged to remove the supernatant;
S13 add 1.0×PBS as wash solution, repeat centrifugation and remove supernatant;
S14 remove the medium and metabolites in it, and centrifuge treatment again to get the bacterial mud.

3. Preparation method of freeze-dried quality control pellets of Escherichia coli as recited in claim 2, S12, S13, and S14) were centrifuged at 8000 rpm for 10 min.

4. Preparation method of freeze-dried quality control pellets of Escherichia coli as recited in claim 1, S4, the freezing process was to freeze the mixture at - 80° C. for 5 h.

5. Preparation method of freeze-dried quality control pellets of Escherichia coli as recited in claim 1, The mass concentration fractions of each component in the lyoprotectant were 2-5% trehalose, 5-8% nonfat dry milk, 2-3% glycerol, 4-5% inorganic salt, 0.05-0.1% antioxidant, and the rest sterile water.

6. Preparation method of freeze-dried quality control pellets of Escherichia coli as recited in claim 5, The mass concentration fraction of each component in the lyoprotectant was: 2% trehalose, 8% nonfat dry milk, 2% glycerol, 5% inorganic salt, 0.05% antioxidant, and the rest sterile water.

7. Preparation method of freeze-dried quality control pellets of Escherichia coli as recited in claim 5, The inorganic salts are any of sodium glutamate, sodium chloride, sodium sulfate, potassium chloride, ammonium chloride, calcium carbonate.

8. Preparation method of freeze-dried quality control pellets of Escherichia coli as recited in claim 5, The antioxidants are any of vitamin C, vitamin E, sodium sulfite.

9. Preparation method of freeze-dried quality control pellets of Escherichia coli as recited in claim 1, The quality control pellets are used for the quality control of different testing equipment or detection methods after be resuspended by adding normal saline or other solutions that are non-destructive to E. coli activity.
